(19) United States
(12) Patent Application Publication
(10) Pub. No.: US 2006/0293554 A1
(43) Pub. Date: Dec. 28, 2006

(54) Surgical Introducer Apparatus and Methods of Use

(76) Inventor: Bruce S. Crawford, Reno, NV (US)

Correspondence Address: KLARQUIST SPARKMAN, LLP, 121 SW SALMON STREET, SUITE 1600, PORTLAND, OR, 97204, US

(21) Appl. No.: 11/426,551

(22) Filed: Jun. 26, 2006

Related U.S. Application Data
(60) Provisional application No. 60/703,698, filed on Jul. 30, 2005.
(60) Provisional application No. 60/694,148, filed on Jun. 24, 2005.
(60) Provisional application No. 60/730,612, filed on Oct. 27, 2005.
(60) Provisional application No. 60/762,262, filed on Jan. 25, 2006.

(51) Int. Cl. A61F 2/00

(52) U.S. Cl. 600/29

(57) ABSTRACT

Certain embodiments of the disclosure provide a surgical instrument and its methods of use. In particular embodiments, the surgical instrument includes first and second arcuate arms, a coupling section connecting the first and second arms, and a material mounting section. In certain embodiments, the second arm is telescopingly mounted in the first arm. In further embodiments, the first and second arms are hingeably connected. A removable sleeve may be placed over the hinge to prevent the first and second arms from moving with respect to one another. In other embodiments, the first arm includes a locking ridge configured to fit in a mating groove of the second arm. The first arm may be biased towards the second arm. Certain methods of using the surgical instruments include making first and second incisions, inserting the first and second arms into respective first and second incisions, and rotating the surgical instrument.

### CROSS REFERENCE TO RELATED APPLICATIONS

This application claims the benefit of, and incorporates by reference, U.S. Provisional Patent Application Nos. 60/703,698, filed Jul. 30, 2005, 60/694,148, filed Jun. 24, 2005, 60/730,612, filed Oct. 27, 2005, and 60/762,262, filed Jan. 25, 2006.

### FIELD

The present disclosure relates to a surgical introducer apparatus and its methods of use, such as for passing materials, such as graft materials for example, through a section of a living body.

### BACKGROUND

The surgical industry has long provided graft augmented repairs or reconstruction of a wide variety of body tissues. The industry has long sought to develop minimally invasive tools and procedures for performing such repairs or reconstruction.

Examples of such graft-augmented surgical operations include the treatment of pelvic organ prolapse and stress urinary incontinence by use of midurethral sling procedures, such as TVT (Tension Free Vaginal Tape Procedure), TOT (Transobturator Tape Procedure), graft augmented cystocele repair including Perigee, graft augmented rectocele repair, and techniques for vaginal vault suspension (IVS Tunneler, Apogee). These surgical procedures typically involve blind passage of an introducer through the fibromuscular elements of the pelvic floor. In one common technique, a curved or helical introducer is introduced blindly and uni-directionally through various layers of tissue including fibro-muscular layers of the pelvic floor. Generally the tip of the instrument is guided toward the surgeon's finger as it penetrates through the pelvic floor or other tissue.

The applicant has discovered that these types of techniques typically incur the risk of one or more problems. For example, prior techniques are often subject to tip wander. As the surgeon attempts to locate the tip of the introducer through layers of pelvic floor, the movement of the tip of the introducer outside the plane of an arc from the point of insertion to the point of exit exposes the patient to additional risk of injury to nerves, blood vessels, and muscular tissue. For example, when an “inside-out” procedure (Gynecare TVT-O) is performed, there is risk of neurovascular injury because it is often difficult to precisely locate tip penetration laterally. In addition, with any of the “outside-in” approaches to the transobturator tape procedure it is common for the tip of the introducer to deviate from a perfect arc as the operator attempts to locate the tip tactilely.

Tip wander also can contribute to patient discomfort. Such patient discomfort may create issues not only after the operation has concluded and the patient is in recovery, but also during the operation if the surgery is performed with only local anesthetic.

Another potential problem with existing techniques is tip visualization. With “outside in” techniques, it can be difficult to visualize the tip of the introducer within body tissue, such as the vagina (especially with heavier patients), making loading of the graft material particularly difficult.

Torque trauma is another potential problem of prior art techniques. The curved instruments commonly used for IVS and Apogee procedures, for example, can tear through a segment of the pelvic floor if torque is applied as the graft is loaded.

Another problem that can occur with existing techniques is operator injury. Glove penetration and finger trauma can occur when performing, for example, any of the outside-in approaches to TOT. Although the surgical instrument tip may be relatively dull in order to reduce the chance of penetration of the operator's finger, a dulled tip typically requires greater force to penetrate the patient's tissues. This greater force in turn increases the chance that the tip of the instrument will forcefully collide with the operator's finger. The dulled tip also exacerbates the possibility that the surgical instrument will cause greater patient trauma and misdirection when using the instrument to perform the desired surgical procedure.

Increased complexity due to specialized instruments and techniques can be another problem when prior art techniques are used. An assortment of instruments of different shapes and sizes are commonly used to accomplish graft placement at differing points in a surgical operation, such as, for example, along the vaginal axis in a vaginal operation. In such situations, the surgeon must be familiar with a variety of surgical instruments and differing techniques for using each such instrument during an operation. The surgeon's learning curve for new instruments and techniques can be daunting.

Certain surgical techniques employ placement of a catheter. One such technique is bladder drainage, a commonly employed technique for a variety of medical reasons. Drainage of the bladder can be accomplished via a urethral catheter or via a supra-pubic route. The latter is a general technique in which a catheter is inserted through the skin of the midline of the lower abdomen, through the various layers of the abdominal wall, and through the anterior wall of the bladder. Generally a balloon is inflated, or an alternative mechanism is used, in order to retain the catheter within the bladder.

The instruments and methods used to perform placement of a supra-pubic catheter are many and varied. These techniques generally fall into three different classes. One such class is the “outside-in” technique. With this technique, the operator typically fills the bladder with fluid and, while performing cystoscopy, inserts a sharp trochar through the abdominal wall and bladder wall into the lumen of the bladder. The catheter is then inserted either around the trochar or through the center of a hollow trochar into the lumen of the bladder. Commercial products used for this technique include the Vesico Set, by J R Bard, and the Rutner Catheter System, by Cook Surgical.

Another technique is the “inside-out” technique. With this technique, the operator typically inserts an instrument into the bladder via the urethra and forces the tip of the instrument through the anterior wall of the bladder and the various layers of the abdominal wall. Once the tip of the instrument (generally a polyp forceps or similar instrument) has been passed through a small supra-pubic skin incision, a ligature, which is connected to a standard Foley catheter, is attached to the instrument. The instrument is then withdrawn through the urethra, bringing the ligature and the catheter along with the instrument. The ligature is removed form the catheter, and the tip of the catheter is backed up into the bladder as the balloon is inflated.

In the “direct technique,” the operator typically makes a larger incision in the abdominal wall over the bladder, incises the anterior wall of the bladder, and inserts a catheter directly into the lumen of the bladder. The bladder is generally secured around the catheter with a purse string suture; and the catheter generally exits the abdominal wall through a small stab incision separate from the surgical incision.

The applicant estimates that 30,000 to 100,000 supra-pubic catheters are placed in women annually in the United States alone. However, prior techniques can provide a number of disadvantages.

For example, the outside-in technique for supra-pubic catheter placement can include problems such as the use of an expensive kit that (i) is not reusable; and (ii) generally provides a catheter with a small lumen that may be subject to occlusion by blood, or other debris, within the bladder. The outside-in technique also typically requires the use of cystoscopy. In the case of the trochar method, excessive downward force can result in poorly controlled entry into the bladder and injury to the opposite wall of the bladder and ureters.

Potential problems with the inside-out technique include the use of a blunt instrument, which can create poorly controlled penetration of the bladder and abdominal wall. The downward rotation of the handle of the instrument can place excessive torque on the urethra, which can lead to injury of the nerve and fibro-muscular tissues surrounding the urethra. The inside-out technique can also cause inadvertent dilatation of the urethra.

Thus, there are disadvantages to each of these prior art techniques. Although specialized instruments have been designed to facilitate the outside-in approach, the applicant is unaware of any such specialized instrument for the inside-out approach.

It is to be understood that, although this disclosure, including the present Background section, describes various aspects and issues with the described prior apparatus and techniques, embodiments of the present disclosure need not necessarily address or resolve any or all such aspects or issues.

### SUMMARY

In particular embodiments, the present disclosure provides a surgical material introducer and methods of use. In some embodiments, the surgical material introducer has a first arm opposite a second arm, a material introducer mount intermediate one of the first arm and the opposing end of the second arm, and an interconnection section connecting the first arm to the second arm. In certain implementations, at least one of the first or second arms are adapted to penetrate tissue and thereby introduce material into and, if desired, through, the tissue.

In some embodiments, the first arm and second arm are arcuate, and the opposing ends of the first arm and second arm are mounted to be moveable in a plane from an open position distal from each other to a closed, or fixed, position adjacent, touching, or overlapping each other. In certain embodiments, one or both of the first and second arms are moveable along an arc, or other predetermined path, from the open position to the closed position.

In particular embodiments, the first and second arm may be secured in a particular configuration, such as in a closed, or partially closed, position. The first and second arms may be secured by placing a groove on the first or second arm and a mating protrusion on the other arm. Alternatively, the arms can be secured in a position by threading material, such as graft or ligature material, through mating slits on the first and second arms. In a further implementation, the first and second arms are connected by a hinge joint. In particular examples, a sleeve may be placed over the hinge joint to prevent rotation of the first and second arms relative to one another.

In certain embodiments, the surgical material introducer has a ring, circular, or similar configuration. The surgical introducer may be rotated in position with respect to penetrated tissue in order to introduce material, such as graft material for example, through the tissue. In further examples, the surgical introducer may be used to place a device, such as a catheter.

In certain embodiments, one or both of the opposing ends of the first and second arm include a tissue penetrating segment. In particular implementations, the tissue penetrating segment is conical or beveled. In further implementations, the tissue penetrating section is smooth and blunt. In particular examples, an end opposite the tissue penetrating section of an arm has a flange or collar adapted to abut tissue.

In certain embodiments, the first arm is telescopingly mounted with respect to the second arm. In some embodiments, the first arm may telescopingly slide into a mating passage in the second arm.

The present disclosure also provides methods of introducing a material into a body section by (i) inserting one end of a disclosed surgical instrument into a body section toward a second opposing end of the surgical instrument, and (ii) rotating the surgical instrument through the body section and thereby pulling material secured to the surgical instrument through at least a portion of the body section. In certain embodiments, the one end moves toward the second end along a predetermined arc in a single plane.

In some embodiments, the method includes rotating the surgical instrument from one portion of a body section to a second portion of a body section, thereby pulling material from outside the one portion through the body section and out the second portion. In some embodiments, the material is graft material. In further embodiments the material is a catheter. In certain examples, the method includes rotating the surgical instrument through at least 180 degrees, such as at least 270 degrees. In further examples, the surgical instrument is rotated at least 360 degrees.

In some implementations, the body section includes the vaginal section of a human body. In further implementations, the body section includes an aperture in a bone within a body. In a particular example, the body section is the bladder.

In certain embodiments, the method includes placing a first arm of a surgical instrument within a vaginal incision and advancing that arm through the layers of the pelvic floor musculature at the position selected to be the fixation point for a biological or synthetic graft. A second, opposing arm of the surgical instrument is advanced through a separate incision until the end of the first arm is secured in a closed position at least relatively adjacent the end of the second arm. The surgical instrument is rotated to bring the ligature or graft into one incision and out another. The ligature or graft is removed from the surgical instrument so that it remains in position between the two incisions.

In some embodiments, the method includes placing a first arm of a surgical instrument, such as a disclosed surgical instrument, into a vaginal incision and advancing a second arm of the surgical instrument through a second incision, such as an incision outside the vagina. In particular examples, the second incision is a groin incision, such as an incision over the inferior pubic ramus. In more particular examples, the method is a mid urethral sling placement via a transobturator approach, a transobturator tape procedure, or a transobturator sub-urethral sling procedure. In further examples, the second incision is a supra-public incision, such as during a mid-urethral sling placement via a retropubic operation. In yet further examples, the second incision is a prepubic incision, such as during a prepubic operation.

In particular embodiments, the material introducer instrument and methods can provide a safer, more accurate, and simpler method of passing a ligature through a body section. The material introducer instrument also can be relatively economical to manufacture and use.

In further embodiments, the present disclosure provides a catheter introducer. In a particular embodiment, the catheter introducer is a ring supra-pubic catheter introducer, an instrument designed to allow surgeons or other operators to introduce a catheter using the inside-out approach without the disadvantages of a linear, blunt instrument. The ring supra-pubic catheter introducer includes a telescoping concentric ring assembly. The introducer also includes a semicircular hollow external element through which a semicircular solid element slides.

The telescoping concentric ring assembly and the semicircular hollow external element, in certain implementations, have at least substantially the same radius. In particular examples, the radius is about 5 cm. In further examples, the hollow element spans through an arc of 180 degrees about the center of the radius, such as approximately 270 degrees about the center of the radius.

The forward end of the hollow element is blunt and smooth, in certain examples. In a more particular example, the blunt, smooth forward end is designed for a traumatic insertion through the urethra. In some implementations, the opposite end of the hollow element contains an external collar, such as a collar designed to abut the external urethral meatus. In particular examples, the forward end of the solid element is sharpened, such as to facilitate easy penetration of the anterior wall of the bladder and the abdominal wall. In yet further examples, the forward end of the solid element also contains an eye hole that can accept a ligature.

The opposite end of the solid element extends out of the back end of the hollow element and can be used by the operator to advance the tip of the solid element through the forward end of the hollow element and through the bladder wall and the abdominal wall. In the “open position” the forward (typically sharp) end of the solid element thus resides within the lumen of the hollow element.

It is to be understood that this is a brief Summary of some aspects of the present disclosure. This summary is not exhaustive, and many additional aspects will become apparent as this specification proceeds. In addition, disclosed embodiments need not include or satisfy all such aspects, nor need they necessarily address the problems or issues set forth in the Background or elsewhere in this specification.

### BRIEF DESCRIPTION OF THE DRAWINGS

The preferred embodiments are shown in the accompanying drawings wherein:

FIG. 1 is side elevational view of a disclosed surgical instrument in an open position;

FIG. 2 is a side elevational view of the surgical instrument of FIG. 1 in a closed position;

FIG. 3 is a partial exploded view of the ligature coupler section of FIG. 1, illustrating the ligature coupler in a unlocked position;

FIG. 4 is a partial exploded, partially cross-sectional view of the ligature coupler section of FIG. 2, illustrating the ligature coupler in a locked position;

FIG. 5 is a detailed view of the surgical instrument of FIG. 1 in the closed position with ligature penetrating the ligature coupler;

FIG. 6 is partial cross-sectional view of the ligature coupler of FIG. 5;

FIG. 7 is a perspective view of a disclosed surgical instrument having a hinge joint.

FIG. 8 is a side elevational view of an alternative embodiment of the surgical instrument of FIG. 1.

FIG. 9 depicts a left pelvis incision and periurethral dissection step, in an exemplary method of use of a rotating graft surgical instrument—in this case, the transobturator approach to mid-urethral sling;

FIG. 10 depicts the step of ring arm insertion within the left periurethral dissection to penetrate the pelvic floor, in the method of FIG. 9;

FIG. 11 depicts the step of rotation the solid ring arm into closed position with the solid arm penetrating the laterial incision and with tape loaded onto the solid ring arm, in the method of FIG. 9;

FIG. 12 depicts the step of rotating the closed ring instrument through 360 degrees, in the method of FIG. 9;

FIG. 13 depicts the step of ring removal with the tape pulled through the vaginal incision and out the lateral incision, in the method of FIG. 9;

FIG. 14 depicts the step of hollow ring arm insertion within the right periurethral dissection to penetrate the pelvic floor, in the method of FIG. 9;

FIG. 15 depicts the step of rotating the solid ring arm into closed position with the solid arm penetrating the laterial incision and with a second tape loaded onto the solid ring arm, in the method of FIG. 9;

FIG. 16 depicts the step of rotating the closed ring instrument through 360 degrees, in the method of FIG. 9; and

FIG. 17 depicts the step of ring instrument removal, with the second tape pulled through the left vaginal incision and out the left lateral incision, in the method of FIG. 9.

### DETAILED DESCRIPTION OF THE PREFERRED EMBODIMENTS

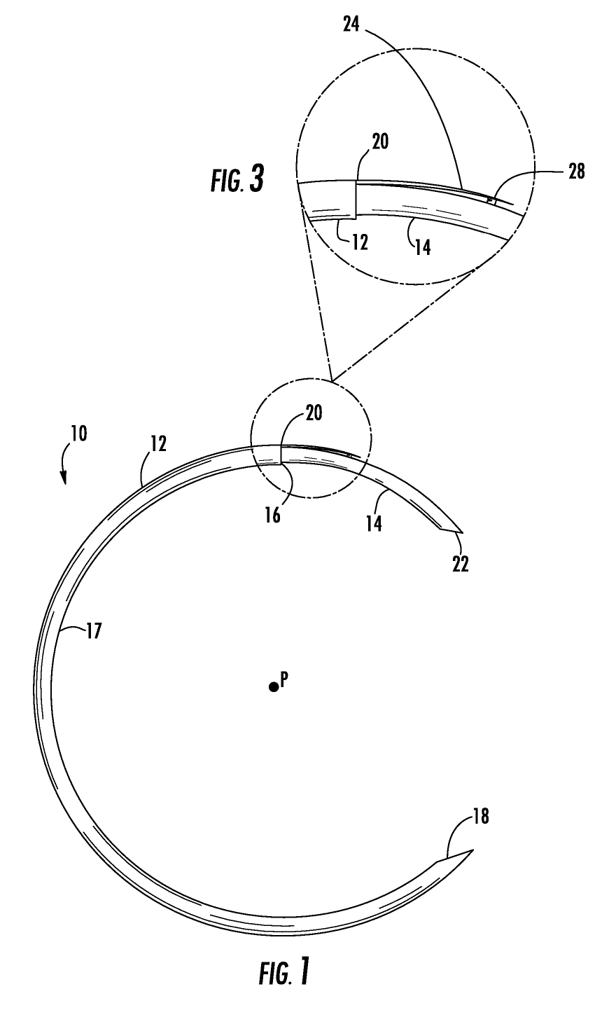

With reference to FIG. 1, one embodiment of a rotating graft surgical instrument, generally 10, has a first cylindrical, outer arcuate arm 12 and a second cylindrical, inner arcuate arm 14 penetrating an interior passage or lumen 16 in the outer arcuate arm 12. The outer arcuate arm 12 extends through an arc of approximately 210-230 degrees (i) about the radial center point P of the outer arcuate arm 12, (ii) in a circle surrounding the outer periphery of the arcuate outer arm 12 about center point P. In particular examples, the diameter of the circle is 8-16 cm. The outer arcuate arm 12, in such examples, may have a side-wall 17 thickness of approximately 0.1-0.5 mm surrounding the interior passage 16, which has a cross-sectional diameter of slightly larger than 3 mm. The inner 14 and outer 12 arms may have differing dimensions, however. The dimensions may be selected based on the application or surgical operation involved.

The arcuate outer arm 12 has a needle end 18 opposite a flattened open end 20. The needle end 18 is pointed for penetration of body tissue, such as, in one example, the fibromuscular tissue of the pelvic floor or the bladder. The interior passage 16 in the open end 20 admits the arcuate inner arm 14 and allows it to slidably telescope into and out of the interior passage or lumen 16 of the arcuate outer arm 12.

The arcuate inner arm 14 is a semi-circular solid member extending through an arc of approximately 240-260 degrees about radial center point P. The diameter of the circle surrounding the outer periphery of the inner arm 14 is approximately 8-16 cm, depending on the version of the instrument produced and the size of the mating arcuate outer arm 12. In particular examples, the inner arm 14 has an outer cross-sectional diameter of 3 mm and a cross sectional shape, radius, and arc that allow it to slide with the lumen 16 of the outer arm 12.

The inner arm 14 has a needle (or sharpened or pointed) end 22 opposite a contained end (not shown). The needle end 22 may be used for penetration of body tissue. The contained end is held within the outer arm 12 by the coupler arm 24 that will prevent the contained end of inner arm 14 from being fully removed from the outer arm 12. In further implementations, the contained end is held in place by a wider section of the inner arm 14 that abuts an internal collar (not shown) of the outer arm 12. The tips 18, 22 can be made with an outside bevel, inside bevel, or with a “pencil tip point” and may be conical or angular. When the tips 18, 22 are beveled, the length of the bevel can be long or short

With reference now to FIG. 2, a curved or arcuate coupler 24 extends from the open end 20 of the outer arm 12 toward the needle end 22 of the inner arm 14. The arcuate coupler 24 is biased radially inwardly to abut the outer periphery of the inner arm 14. When the inner arm 14 is in the “open position”—with the needle end 22 of the inner arm 14 distal from the needle end 18 of the outer arm 12 as shown in FIG. 1—the curved coupler 24 has a thickened ligature mounting section 28 that slidably abuts the convex outer periphery of the inner arm 14.

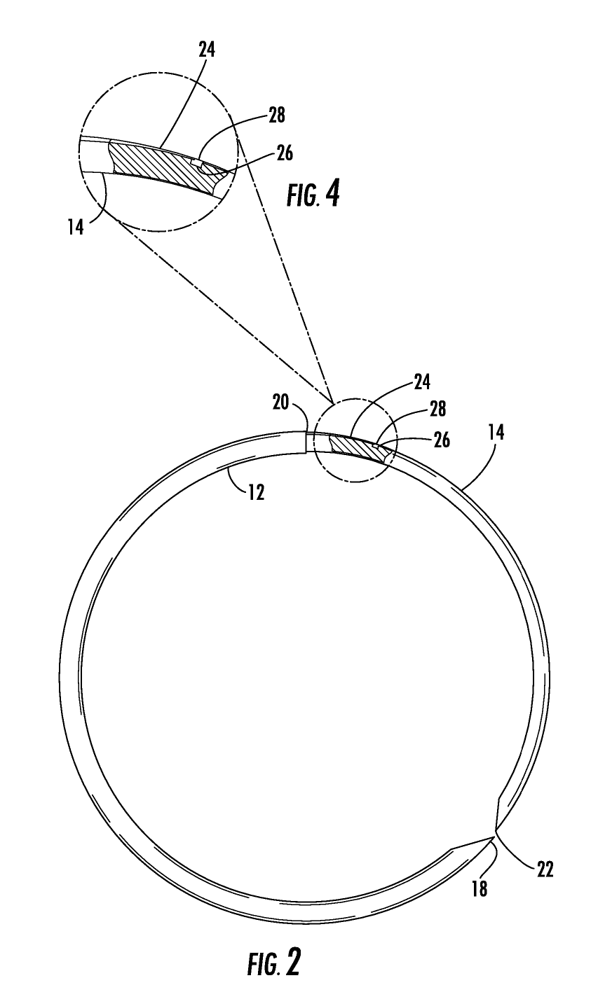

With reference to FIGS. 3 and 4, the arcuate inner arm 14 may slidably telescope outwardly from the mating outer arcuate outer arm 12 so that the needle end 22 of the inner arm 14 is in at least relatively close proximity, such as within 1 mm or less, of the needle end 18 of the outer arm 12. As shown in FIG. 4, in this closed ring position the ligature coupler 24 is biased radially inwardly, or “pre-tensioned”, to lockingly penetrate a mating coupler catch, groove, or detent 26 penetrating the external periphery of the inner arm 14 adjacent but external of the open end 20 in the outer arm 12. In this locking position, the coupler 24 secures the outer arm 12 and inner arm 14 in the closed ring position and abuts the convex external periphery of the inner arm 14 in order to minimize friction between (i) the inner arm 14 and the associated coupler 24, and (ii) any tissue or other material that these structures may contact during use of the surgical instrument 10. In particular implementations, the inner arm 14 and outer arm 12 are flush when the coupler 24 secures the instrument 10 in a locked position.

The coupler mechanism 24 can have various different designs. For example, the coupler can have double coupler arms, a collar screw type (collar and fastener) coupler, a detachable clip-on type coupler, a tip coupler (a component that holds the ring in a closed position by joining the tip when they come together as the ring is closed), or a pin coupler (where a pin is inserted through both arms 12, 14 of the instrument 10 once the ring is completely closed). In further examples, the tip 18 of the hollow outer arm 12 can open and allow the solid inner arm 14 to enter the outer arm 12 as the instrument is closed or it can be sealed to prevent entry of the solid arm 12. Alternatively, the needle 18, 22 of one arm may be adapted to come into contact but not penetrate the opposing needle end of the other arm.

In further examples, the coupler mechanism includes an outwardly biased locking member extending from the secured end of the inner arm 14 and having a radially outwardly extending locking nipple (not shown). The locking nipple can lockingly penetrate a mating aperture in the outer arm 12. The locking nipple can be depressed inwardly into the lumen in the outer arm to unlock the arms with respect to each other.

Alternatively, the coupler 24 can have an arcuate configuration adapted to slide along the outer periphery of the inner 14 and outer arms 12 with opposing coupler arms biased toward the axial center of the coupler to snap into position within a mating narrowed section on one or both of the inner 14 and outer 12 arms. To unlock the surgical instrument, such a coupler 24 can be manipulated to force the opposing coupler arms outwardly from their axial center and thereby away from interlocking contact with the mating narrowed section(s).

As an alternative to having a latch type coupling mechanism, the ring can be secured into a locked position by threading a graft, ligature, or other material, through elongated apertures in two opposing walls of the hollow element that align with an elongated aperture in the solid element at a point where the hollow element and the solid element overlap while the ring is in a full closed position. This will maintain the ring in a stable “locked” closed position as the ring is rotated. The threaded slit coupler also provides a means of securing a graft or other material to the instrument 10.

In particular examples, the approximate dimensions of the elongated apertures in the hollow and solid elements are: length 0.45″, width 0.025″. The slit may be positioned approximately two inches from the blunt end 20 of the hollow element 12.

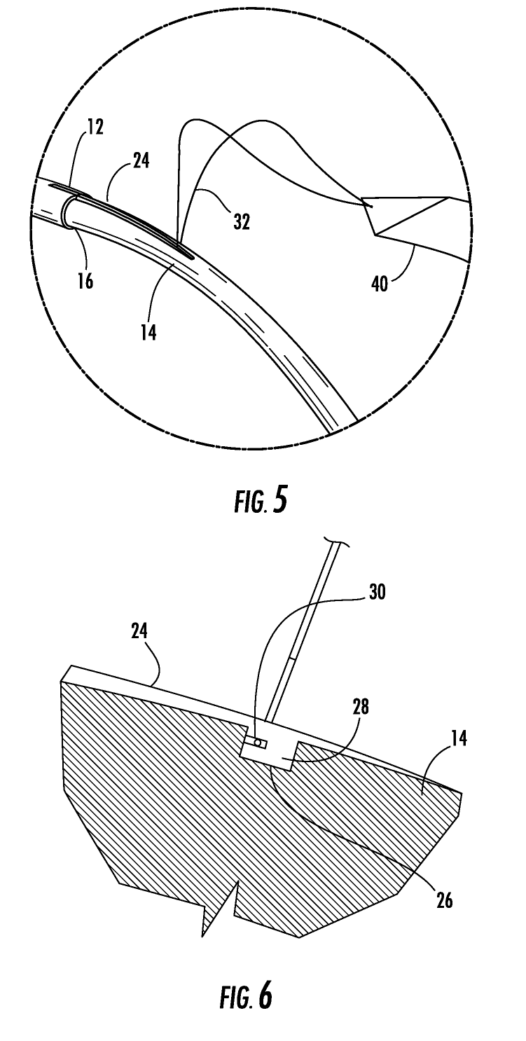

With reference to FIGS. 5 and 6, the coupler 24 has a ligature mounting structure 30, such as a slot generally concentric with the arc of the ligature mounting section 28. The ligature mounting structure 30 can be formed in other portions of the surgical instrument 10, including in any suitable portion of the outer arm 12 and inner arm 14. In particular examples, the ligature mounting structure 30 may be formed in multiple structures of the surgical instrument 10. In other implementations, the ligature mounting structure 30 is a separate structure that mounts in position with respect to the arms 12, 14. The coupler 24, or arm inter-locking structure, may be separate from the ligature mounting structure 30.

Ligature 32 can be inserted into the ligature mounting section 30 when, as shown in FIG. 6, the ligature mounting section 30 is external of the mating coupler catch 26 as shown in FIG. 6. When, as shown in FIG. 5, the ligature mounting section 28 penetrates the mating coupler catch 26, the ligature 32 is secured in position in the ligature mounting section 30. The ligature 32 can be used to pull upon and elevate the coupler arm 24 out of the mating coupler catch 26 in outer periphery of the inner arm 14. Of course, the ligature mounting section 30 may be adapted to provide for mounting of other types of material, not only those that can be used to bring, as shown in FIGS. 5 and 6 a flexible graft material 40 through a tunnel (not shown in FIGS. 1-6) created by the surgical instrument 10. For example, the surgical instrument 10 may be used to place a catheter.

The ligature mounting section 28, or “tail” apparatus, can be used to attach graft material, or other material to be introduced, to the surgical instrument 10. The tail apparatus may be designed differently so as to accommodate different graft materials. For example a piece of biological graft material might be sutured and the suture threaded through a loop element at a desired location along the inner arm 14, the outer arm 12, or intermediate the inner 14 and outer 12 arm, such at the end of the ligature mounting section 28. When the coupler 24 is a hinge, the material may be attached through the roll pin in the hinge mechanism. Various different designs of the tail apparatus could be used depending on the specific brand of praline polypropylene tape that might be used. The tail element can be attached to the ring on the coupler arm, the internal arm, or the external arm in such a way that that will not interfere with the opening, closing, or locking of the surgical instrument 10. In particular implementations, the ligature mounting section 28 is made of a flexible, smooth material with a tensile strength sufficient to allow an amount of traction on the tail needed to pull the tape through an incision, such as a vaginal incision, and out through a second incision. The ligature or graft can be loaded onto the ring in different ways as well. For example, the ligature or graft may be mounted through a passage, or on a ring-mounting clip (or loop),

The surgical instrument 10, including the inner arm 14 and the outer arm 12, may be made of stainless steel or other rigid and strong material, such as metal, plastic, resin, or composite. These components are typically sterilized prior to use in a surgical procedure, whether involving a human, animal, or other living entity. In this regard, the ring instrument 10, components thereof, may be disposable or reusable.

The inner arm 14 and outer arm 12 may also have configurations other than circular and be mounted in other ways with respect to each other. For example, arcuate portions of the inner arm 14 and the outer arm 12 need not be circular. In addition, in certain implementations, the inner arm 14 and the outer arm 12 are linear, or include linear portions.

The configuration and mounting structure can thus be modified to provide for the desired movement of one end of the instrument with respect to the other end, in order to procure the desired result of having one end automatically move to a given, predetermined position with respect to the other end during the surgical procedure. The configuration may thus be altered while still providing, if and as desired, motion of one arm with respect to the other arm in a single predetermined plane, in an arc within the plane, or in another path within the plane.

The surgical instrument may have yet additional structure. For example, the instrument may have a central section intermediate the first and second arms. The central section may be arcuate or have another shape as desired in order to provide a desired form of instrument and associated movement of the instrument during an operation.

FIG. 7 illustrates a further embodiment of a surgical instrument 100. The surgical instrument includes a first arcuate cylindrical arm 108 and a second arcuate cylindrical arm 112. The first arcuate arm 108 includes a sharpened end 118 and a mounting end 122. The mounting end 122 defines a ligature mounting aperture 128 extending through the first arcuate arm 108. An arcuate cylindrical mounting arm 132 extends from the mounting end 122 of the first arcuate arm 108. The end 136 of the mounting arm 132 opposite the mounting end 122 is forked. Each arm 138 of the fork has an aperture 140 formed therein.

The second arcuate arm 112 also has a sharpened end 146 and a mounting end 150. An arcuate cylindrical mounting arm 156 extends from the mounting end 150 of the second arcuate arm 112. The hinge end 160 of the mounting arm 156 is a flattened half obround defining an aperture 164.

The hinge end 160 of the mounting arm 156 is configured to be received within the arms 138 of the forked end 136 of the mounting arm 132. The apertures 140 in the arms 138 align with the aperture 164 in the end 160. A pin 170 may be inserted into the apertures 140, 164 in order to secure arms 108 and 112 together. The ends 136, 160 thus form a hinge joint 174 that allows the first arm 108 and the second arm 112 to be rotated in a plane with respect to one another.

The instrument 100 may include an arcuate generally cylindrical sleeve or collar 180. The sleeve 180 has a longitudinal gap 184. The cylindrical sleeve 180 may be formed from a flexible material, such as surgical grade polymers. In some examples, the sleeve 180 is made of metal or a hard resin. The longitudinal gap may be placed over the hinge 174 in order to prevent movement of the arms 108, 112 relative to one another. In particular examples, the sleeve 180, when inserted onto the device 100, is flush with the arms 108, 112 (that it, the diameter of the portion of the device with the sleeve 180 is at least substantially the same as the diameter of the arms 108, 112). In some implementations, the sleeve 180 does not have a slit. In further implementations, the sleeve 180 does not need to be removed from the instrument 100, but can be slid off and on the hinge 174 to selectively secure the arms 108, 112. The hinge area 174 may be recessed compared to the arms 108, 112 in order to limit movement of the sleeve 180.

In further implementations, the arms 108, 112 or mounting arms 132, 156 may be shaped differently. For example, arcuate portions of the arms 108, 112, 132, 156 need not be circular. In addition, in certain implementations, the arms 108, 112, 132, 156 are linear, or include linear portions. For example, the instrument 100 may have a V-shape.

In further implementations, one or more of the arms 108, 112 are hollow and the mounting arms 132, 156 may be slidable within a respective arm 108, 112. In this way, the configuration of the device 100 may be varied. The ligature mounting section 128 and ends 118, 146 may be varied as described above for the instrument 10 of FIG. 1. In addition, the hinge joint 174 may be replaced by other suitable coupling sections, such as described for coupler 24 of FIG. 1. In a particular example, the pin 170 and ligature mounting section 128 are omitted and ligature is inserted through the apertures 140, 164, thus attaching the ligature to the device 100 and securing the arms 108, 112.

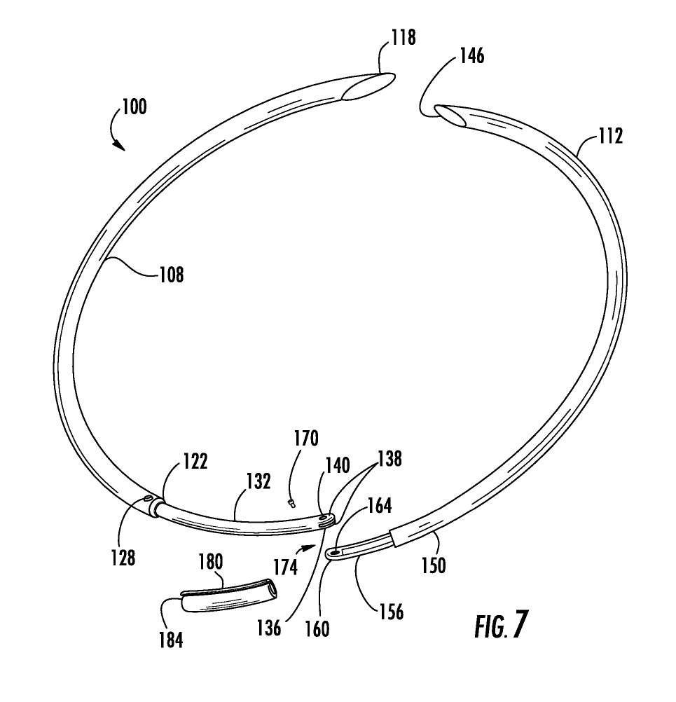

FIG. 8 illustrates another surgical instrument 200. The surgical instrument 200 is generally similar to the instrument 10 of FIG. 1. The surgical instrument 200 has an outer, hollow arcuate arm 208 and a solid inner arcuate arm 212. At least a portion of the inner arm 212 is movable within the outer arm 208. The outer arm 208 includes a smooth, blunt end 220 through which the inner arm 212 may extend. The opposing end 224 of the outer arm 208 includes a flange or collar 230. The end 236 of the inner arm 212 is sharpened, such as to penetrate tissue. The end 236 defines an aperture 240 through which surgical material may be threaded.

The instrument 200 may be use in procedures such as introducing a catheter into the bladder. The smooth end 220 of the outer arm 208 may be inserted into the urethral meatus until the collar 230 abuts the urethral meatus. The inner arm 212 (a portion of which may extend from the end 224 of the outer arm 208) may then be advanced so that the sharpened end 236 penetrates tissue.

The surgical instruments disclosed herein can be used in a wide variety of surgical operations, including on animals or other non-human living entities.

### EXAMPLE 1

### Mid Urethral Sling Placement via a Transobturator Approach

A female human patient is placed in a modified lithotomy position. Adequate anesthesia is established, and the incision site(s) is determined. The incision site(s) is prepared and draped, as appropriate.

As shown in FIG. 9, a 0.5 cm skin incision is made over the inferior pubic ramus at the level of the base of the clitoris. A one centimeter incision is made in the epithelium of the anterior vagina wall beginning approximately 1 cm from the external urethral meatus and extending cephalad. A periurethral dissection is then carried out, toward the groin incision, bluntly until the medial edge of the inferior pubic ramus is reached.

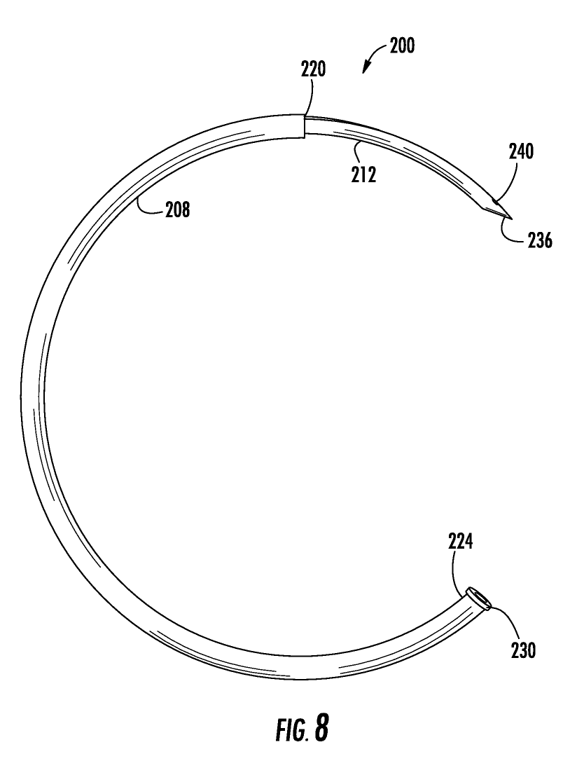

As shown in FIG. 10, with a ring graft introduction surgical instrument in an open position, the needle end of the outer ring arm is inserted into the left periurethral dissection. This same needle end is advanced into the dissection while the operator, palpating through the vaginal epithelium, guides the tip up to the medial edge of the inferior pubic ramus. This same needle end is then advanced around the inferior pubic ramus and through the fibro-muscular layers of the pelvic floor as it enters the obturator foramen. A ligature is loaded into a mating slot in a ligature mounting section or other mounting structure for the ligature.

With reference now to FIG. 11, the outer arm is then grasped with the opposite hand and held steady while the inner arm is advanced out of the lumen of the hollow arm. The opposing needle end of the inner arm is guided into the left groin incision made previously. The inner arm is advanced through the subcutaneous fat, obturator externus, and obturator membrane, as it approaches the tip, or needle end, of the outer arm. As the needle end of the inner arm approaches the opposing needle end of the outer arm, the ligature mounting section slides along the outer periphery of the inner arm until it penetrates the mating coupler catch in the outer surface of the inner arm. The ring instrument is thereby secured in a closed position with the opposing needle ends adjacent each other.

With reference now to FIG. 12, the ring instrument is rotated 360 degrees outwardly, bringing the ligature through the vaginal incision and out through the left lateral incision. As an alternative, an inward rotation can be performed, bringing the tip of the inner arm out through the vaginal incision, at which point the ligature or graft can be loaded onto the tip of the inner arm. With the latter method, the ring instrument would be unlocked or opened and removed bringing the ligature or graft through the vaginal incision and out of the groin incision.

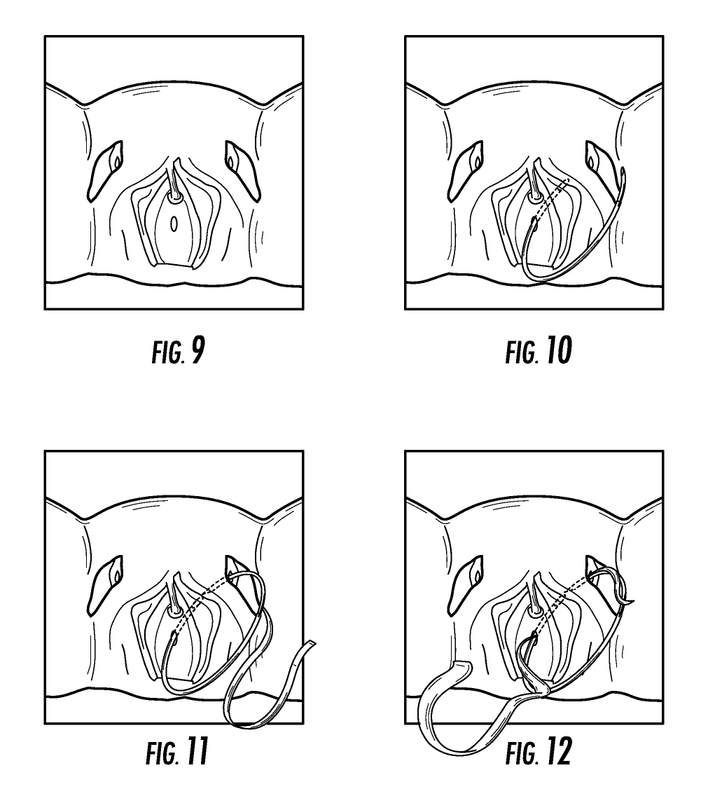

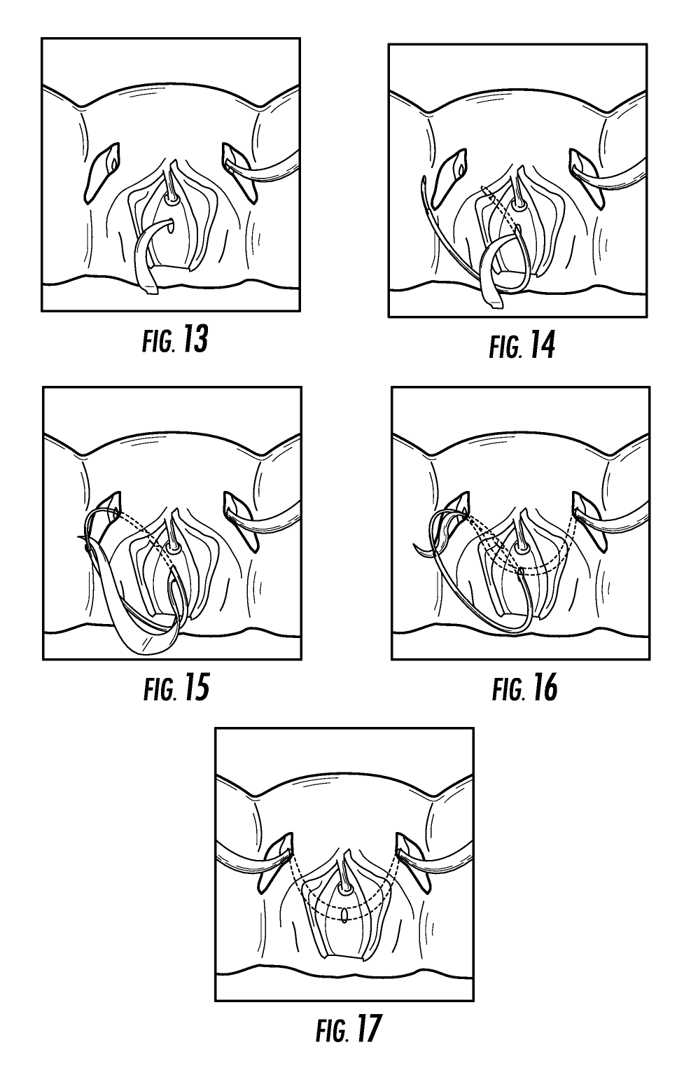

With reference to FIG. 13, following the 360 degree rotation, the ring is unlocked and removed, bringing the ligature or tape through the vaginal incision and out through the groin incision. The ring instrument is unlocked by either elevating the ligature mounting section on the coupler arm out of the mating coupler catch in the inner arm or by applying sufficient force to raise the ligature mounting section from contact with the inner arm. Alternatively the ring could be rotated 180 degrees and the opposing needle ends of the inner and outer arm may be brought through the lateral incision, at which point the inner arm can be pushed into the mating interior passage in the outer arm until the ring instrument is removed from the patient completely and the ligature is positioned into the vaginal incision and out through the lateral incision.

With reference now to FIGS. 14-17, the above-described procedure can be repeated on the contralateral side, using the opposite end of the graft so that the graft is positioned under, and loosely applied to, the underside of the mid-urethra. The incisions can then be closed in a fashion well known to those skilled in the art.

### EXAMPLE 2

### Midurethral Sling Placement via a Retropubic Operation

After establishing anesthesia, surgical preparation, and draping the patient in a modified lithotomy position, the sites for the vaginal and suprapubic incisions are identified and marked. A one centimeter incision is made in the sub urethral vaginal epithelium and a small periurethral dissection is carried out bilaterally.

With the ring instrument in an open position, the needle end of the outer arm is inserted into the left periurethral dissection. The periurethral fibro-muscular tissue is penetrated by the needle end of the outer arm. The needle end of the inner arm is inserted into the suprapubic incision and advanced through the subcutaneous fat and fibromuscular elements of the abdominal wall until the tip of the inner arm approaches the needle end of the outer arm. As the opposing needle ends come together, the ligature mounting section or other inter-locking section on the coupler arm penetrates the mating catch on the inner arm, locking the ring instrument in the closed position. Alternatively the needle end of the inner arm could be inserted into the periurethral dissection and the outer arm brought through the suprapubic incision.

The ring instrument is rotated 360 degrees, bringing ligature into the vaginal incision and out through the suprapubic incision. The ring instrument is unlocked, opened, and removed by telescoping the inner arm back into the outer arm. As the ring instrument is removed, the ligature or graft is left entering the vaginal incision and exiting out through the suprapubic incision.

The above-described steps are repeated on the contralateral side using the opposite end of the graft, bringing the graft up under, and loosely applied to, the urethra. The vaginal and suprapubic incisions are closed in the usual fashion.

### EXAMPLE 3

### Prepubic Operation

Following anesthesia, the patient is placed in a modified lithotomy position. The surgical site is prepped and draped, and the surgical incisions are marked. A one centimeter incision is made in the sub urethral vaginal epithelium.

With the ring instrument in an open position, the needle end of the outer arm is inserted into left periurethral dissection and advanced laterally and anteriorly over (superficial to) the inferior pubic ramus, so that the needle end of the outer arm comes to rest at approximately the level of the base of the clitoris. The needle end of the inner arm is then inserted into the incision over the pubic bone and advanced toward the needle end of the outer arm. As the opposing needle ends approach each other, the ligature mounting section on the coupler arm is biased into the mating catch on the inner arm, locking the ring instrument in the closed position.

The ring instrument is rotated 360 degrees, bringing the ligature or graft into the vaginal incision and out through the prepubic skin incision. The ring instrument is removed, and the graft is pulled through the vaginal incision and out through the prepubic skin incision. The procedure is repeated on the contralateral side.

### EXAMPLE 4

### Graft Augmented Rectocele Repair and Vaginal Vault Suspension

After induction of anesthesia the patient is placed in a modified lithotomy position, prepped, and draped in the usual fashion. The surgical incision sites are marked. The epithelium of the posterior vaginal wall is divided in the midline and dissected off the underling rectum broadly out to the pelvic floor musculature laterally and up to the ishial spines bilaterally.

The needle end of the outer arm is inserted into the left perirectal dissection and into the fibro-muscular layers of the pelvic floor immediately anterior to the ishial spine. The inner arm is advanced into the left perianal incision through the ishial-rectal fossa and up to the needle end of the outer arm. As the needle end of the inner arm approaches the needle end of the outer arm, the ligature mounting section on the coupler arm penetrates the mating catch on the inner arm so that the ring instrument is in a closed and locked position.

The ring instrument is rotated 360 degrees, simultaneously pulling the associated ligature or graft into the vaginal incision and out through the perianal incision. The ring instrument is removed, and the graft is pulled through the vaginal incision and out through the perianal incision. The procedure is repeated on the contralateral side.

The graft is attached to the vaginal apex with tacking sutures. If a rectocele repair is to be performed, a specialized graft is used. The graft is sutured to the fascia of the pelvic floor laterally and to the fibro-muscular elements of the perineal body distally. The vaginal epithelium is closed.

### EXAMPLE 5

### Graft Augmented Cystocele Repair

After induction of anesthesia the patient is placed in a modified lithotomy position, prepped, and draped in the usual sterile fashion. The surgical incision sites are marked. The epithelium of the anterior vaginal wall is divided along the midline and elevated off the underlying bladder out to the pelvic floor musculature laterally and up to the ishial spines cephalad.

The needle end of the outer arm is inserted into the left dissection. The needle end of the inner arm is inserted into the fibro-muscular tissue of the pelvic floor immediately anterior to the left ishial spine. The inner arm is inserted into the left lateral groin incision and advanced through the obturator externus, the obturator membrane, and the obturator internus as it approaches the needle end of the outer arm. As the needle end of the inner arm approaches the needle end of the outer arm, the ligature mounting section on the coupler arm penetrates the mating catch on the inner arm, bringing the ring instrument into a closed and locked position.

The ring instrument is rotated 360 degrees, simultaneously pulling the ligature or graft into the vaginal incision and out through the lateral groin incision. The ring instrument is removed, and the graft is pulled into the vaginal incision and out through the left lateral groin incision. The procedure is repeated on the contralateral side.

The needle end of the outer arm is inserted into the left dissection. The needle end of the outer arm is inserted into the fibro-muscular tissue of the pelvic floor at the level of the bladder neck toward the obturator foramen immediately cephalad to the inferior pubic ramus. The needle end or tip of the inner arm is inserted into a separate lateral groin incision and advanced toward the needle end of the outer arm. As the needle end of the inner arm approaches the needle end of the outer arm, the ligature mounting section on the coupler arm penetrates the mating catch on the inner arm, bringing the ring instrument into a closed and locked position.

The ring instrument is rotated 360 degrees, simultaneously pulling the ligature or graft into the vaginal incision and out through the left lateral groin incision. The ring instrument is removed, and the left distal arm of the graft is pulled through the vaginal incision and out through the left lateral groin incision. The procedure is repeated on the contralateral side using the distal right arm of the graft. The graft is tacked to the fascia of the pelvic floor musculature laterally. The vaginal epithelium is closed.

### EXAMPLE 6

### Placement of a Supra-Pubic Catheter

With the ring in an open position, the forward end of the hollow element is inserted into the external urethral meatus. The introducer instrument is rotated forward bringing the forward end of the hollow arm through the urethra and into the bladder lumen. The hollow arm is advanced until the external collar abuts the external urethral meatus. In this position the tip of the hollow arm abuts, and tents up, the inside of the anterior wall of the bladder. The solid arm is then advanced out of the tip of the hollow arm through the anterior wall of the bladder and the various layers of the anterior abdominal wall. Ultimately the tip of the solid arm emerges through a small supra-pubic skin incision.

With the tip of the solid arm above the level of the skin, a ligature is threaded though the eye hole. The solid arm is then withdrawn back into the hollow arm, and the entire introducer instrument is removed from the urethra bringing the ligature through the abdominal wall, into the bladder, and out through the urethra. The opposite end of the ligature is attached to the tip of a standard Foley catheter. The ligature is used to draw the Foley catheter through the abdominal wall, into the bladder, and out through the urethra. The ligature is removed from the tip of the catheter. The tip of the catheter is then drawn back into the bladder lumen as the balloon is inflated.

### EXAMPLE 7

### Transobturator Tape Procedure

Landmarks for the site of the lateral incision are located and a vaginal incision and periurethral dissection are created. With the ring introducer in the full open position, the tip of the solid end is placed into the vagina so as to get the ring into the proper position. With ring in the full open position the tip of the hollow arm is inserted through the groin incision until the obturator membrane is penetrated.

The tip of the solid arm is inserted through the vaginal incision until the ring is in a full closed position as indicated by a color indicator on the hollow arm or visualization of alignment of the “tape insertion” window in the hollow and solid arms. The tape is loaded into the tape insertion window locking the ring in a full closed position

The vaginal sulci is inspected to confirm there is no epithelial perforation. The ring is rotated inside-out bringing the tape into the vaginal incision and out through the lateral incision. The tape is unloaded from the ring (now the ring is unlocked). While holding the solid arm steady the hollow arm is brought out through the groin incision and the ring is completely removed from the patient.

### EXAMPLE 8

### Transobturator Sub-Urethral Sling Procedure

The sub-urethral mucosa is infiltrated and incised per routine for any mid-urethral sling procedure. The lateral incision site is identified using the base of the clitoris, the thigh crease, and the insertion of the adductor longus tendon as landmarks. This site is marked, infiltrated with local anesthetic and a 2-3 mm incision made. A peri-urethral dissection is carried out with scissors up to the medial edge of the inferior pubic ramus as would be done with any of the various techniques for trans-obturator tape placement.

The internal arm of ring introducer is inserted into the left peri-urethral dissection and advanced up to the medial edge of the obturator foramen. Correct placement of the internal arm can be verified by palpation through the vaginal epithelium. It is not necessary for the operator to insert a finger into the periurethral dissection.

Holding the internal arm in the left hand the external arm is advanced into the lateral incision. The external arm is then advanced through the subcutaneous fat, obturator membrane, and obturator internus musculature before finally bringing the ring into a closed and locked position as the ridge(s) on the coupler fall in to the groove(s) on the outside arm.

With this accomplished, the tape is loaded into the receiving end of the ligature or “tail” apparatus attached to the ring. With the tape loaded the ring is rotated 360 degrees bringing the tail into the vaginal incision and out through the lateral incision. The ring is unlocked by elevating the coupler arm so as to elevate the ridges out of the groove(s) in the outside arm. The outside arm is pushed back into the lumen of the inside arm and the ring is withdrawn from the patient dragging the tail through the vaginal incision and out through the lateral incision. The tape is then drawn through the vaginal incision and out through the lateral incision using the tail.

The procedure is repeated on the opposite side using the same ring and the opposite arm of the tape. With the tape in position the tension is adjusted, the lateral ends trimmed, and the incisions closed in a routine manner.

It should be noted that graft passage procedures can be done as described above or can be done by rotating the ring instrument inward, bringing the needle end of the inner arm out through the vaginal incision. With the needle end of the inner arm exposed, the ligature or graft can be loaded onto the ring instrument, and the ring instrument can be rotated in the opposite direction (outwardly), dragging the ligature or tape through the vaginal incision and out through the groin incision.

In addition, graft passage procedures can be done as described above or begun with insertion of the inner arm into the vaginal incision followed by passing the outer or opposing arm through the outside incision. Subsequently, the ring instrument can be rotated with either an inside role or and outside role depending on the position on which the ligature or graft is to be loaded onto the ring instrument.

A ring or similar surgical introducer may be utilized in many surgical procedures other than those described above. For example, other types of surgical operations in which a ring or similar surgical introducer may be used include: the male sling procedure, placement of artificial urethral sphincter, placement of artificial anal sphincter, and orthopedic or general surgical procedures requiring passage of materials around bony structures.

It can thus be seen that, in at least certain embodiments disclosed herein such as the embodiments of FIGS. 1-8, the surgical instrument and its method of use can provide graft material placement with a number of advantages:

One advantage of the disclosed instrument is accuracy. Because the point of fixation is determined by the direct placement of the needle end of one arm with respect to the other arm, fixation is more accurate. For example, in the methods set forth above, the point at which the graft material penetrates the medial surface of the pelvic floor fibromusculature determines the fixation point for the graft. With regard to the midurethral sling via the transobturator approach it is common for operators to place the graft too low with respect to the obturator foramen, the pelvic floor and the vaginal lumen. This occurs because the fibromuscular layers of the pelvic floor are the last layers penetrated when performing an outside-in procedure. With at least certain of the surgical instrument embodiments disclosed herein, the operator may utilize an inside-out approach by directly selecting the optimal fixation point for the graft with one arm of the surgical instrument, while at the same time preserving the safety advantages of an outside-in approach with the other arm of the surgical instrument.

Precision is another advantage that may be provided by disclosed instruments. In at least certain embodiments, the end of the outer or first arm is constrained to move toward the end of the inner or second arm in a single predetermined plane, and in some embodiments, along a predetermined arc in such a plane, thus reducing or even eliminating wander of the end of a body penetrating arm or, if desired, the opposing ends of both arms as they penetrate a body section.

The disclosed embodiments can be safer to use than prior instruments and techniques. For example, because tip wander is reduced or eliminated, the risk of nerve or vascular injury is reduced. In addition, because the instrument is self-guided in a predetermined path, there is little or no risk of injury to the operator's finger, for example.

Disclosed instruments also have the advantage of adaptability. For example, a single introducer design can be used for many and possibly all graft-augmented repairs performed by a given operator or facility. In addition, the two-dimensional introducer structure, particularly the ring or circular introducer structure, has an aesthetic appearance that can be more readily accepted by surgeons performing these procedures. The process of education and use of the introducer instrument can therefore be more efficient and less daunting. Surgical operations with the instrument can be more reliable, more efficient, and less subject to error.

It is to be understood that the foregoing is a detailed description of preferred and other embodiments. The foregoing is therefore illustrative of certain embodiments and not limiting of the scope of the disclosure.

I claim:

1. A surgical instrument comprising:
(A) an arcuate first arm;
(B) a second arcuate arm moveably mounted with respect to the arcuate first arm, wherein the arcuate second arm may be positioned in at least relatively close proximity to the arcuate first arm; and
(C) a material introducer mount in communication with the first or second arm;
whereby rotation of the surgical instrument through a penetrated body section simultaneously rotates the material introducer mount through the penetrated body section.

2. The surgical instrument of claim 1, wherein the arcuate first arm comprises a cavity and at least a portion of the arcuate second arm is slidably positioned within the cavity.

3. The surgical instrument of claim 2, wherein the arcuate first arm has first and second ends, a portion of the arcuate second arm positioned within the cavity comprising a contained end, an opposing end of the arcuate second arm being extendable from the first end of the arcuate first arm and received by the second end of the arcuate first arm.

4. The surgical instrument of claim 2, wherein the arcuate first arm has first and second ends, the at least a portion of the arcuate second arm positioned within the cavity comprising a contained end, an opposing end of the arcuate second arm being extendable from the first end of the arcuate first arm, the material introducer mount formed on the opposing end of the arcuate second arm.

5. The surgical instrument of claim 4, the material introducer mount comprising a slot defined by the opposing end of the arcuate second arm.

6. The surgical instrument of claim 4, the material introducer mount comprising a loop.

7. The surgical instrument of claim 4, the arcuate first arm comprising a protruding lock member and the arcuate second arm defining a lock slot configured to receive the lock member.

8. The surgical instrument of claim 7, wherein the protruding lock member is formed on a tapered portion of the arcuate firm arm and the tapered portion of the arcuate first arm is biased towards the arcuate second arm.

9. The surgical instrument of claim 4, the arcuate second arm comprising a protruding lock member and the arcuate first arm defining a lock slot configured to receive the lock member.

10. The surgical instrument of claim 9, wherein the protruding lock member is formed on a tapered portion of the arcuate second arm and the tapered portion of the arcuate second arm is biased towards the arcuate first arm.

11. The surgical instrument of claim 2, wherein the arcuate first arm has first and second ends, the at least a portion of the arcuate second arm positioned within the cavity comprising a contained end, an opposing end of the arcuate second arm being extendable from the first end of the arcuate first arm, the material introducer mount comprising slots formed on the opposing end of the arcuate second arm and the second end of the arcuate first arm.

12. The surgical instrument of claim 2, wherein the arcuate first arm has first and second ends, the at least a portion of the arcuate second arm positioned within the cavity comprising a contained end, a pointed opposing end of the arcuate second arm being extendable from the first end of the arcuate first arm.

13. The surgical instrument of claim 2, wherein the arcuate first arm has first and second ends, the at least a potion of the arcuate second arm positioned within the cavity comprising a contained end, an opposing end of the arcuate second arm being extendable from the first end of the arcuate first arm, the arcuate first arm being pointed.

14. A surgical method comprising:
making a first incision;
making a second incision;
inserting the arcuate first arm of the surgical instrument of claim 1 into the first incision;
inserting the arcuate second arm of the surgical instrument of claim 1 into the second incision; and
rotating the surgical instrument of claim 1.

15. The surgical method claim 14, furthering comprising attaching a surgical material to the material introducer mount prior to rotating the surgical instrument of claim 1.

16. The surgical method of claim 14, wherein the first incision is a vaginal incision and the second incision is a groin incision.

17. The surgical method of claim 14, wherein the first incision is a vaginal incision and the second incision is a supra-pubic incision.

18. The surgical method of claim 14, wherein the first incision is a vaginal incision and the second incision is pre-pubic incision.

19. A surgical method comprising:
inserting the surgical instrument of claim 1 into the external urethral meatus of a subject;
inserting a first end of the second arcuate arm of the surgical instrument of claim 1 through the bladder of the subject;
inserting the first end of the second arcuate arm through a supra-public incision;
mounting a surgical material to the material introducer mount of the surgical instrument of claim 1; and
rotating the surgical instrument of claim 1 to remove the first end of the second arcuate instrument from the external urethral meatus.

20. A surgical instrument comprising:
first arm means for penetrating a tissue of a subject by moving the first arm means through an arcuate path;
second arm means for penetrating a tissue of the subject by moving the second arm means through an arcuate path;
interconnection means for connecting the first arm means to the second arm means so that the first arm means is moveable with respect to the second arm means; and
mounting means for attaching a surgical material to the surgical instrument so that arcuate movement of at least a portion of the surgical instrument moves the surgical material through the tissue of the subject.

21. The surgical instrument of claim 20, further comprising locking means for securing the first arm means relative to the second arm means.

22. A surgical method comprising:
an incision step for making a first incision;
an incision step for making a second incision;
an insertion step for inserting the surgical instrument of claim 20 in the first incision; and
a rotating step for rotating the surgical instrument of claim 20 through the first and second incisions.

23. The surgical method of claim 22, further comprising a mounting step for mounting a surgical material to the mounting means of the surgical instrument of claim 20.
